(19) United States
(12) Patent Application Publication
(10) Pub. No.: US 2023/0301550 A1
(43) Pub. Date: Sep. 28, 2023

(54) REAL-TIME HUMAN ACTIVITY RECOGNITION ENGINE

(71) Applicant: VirtualBeam, Inc., San Francisco, CA (US)

(72) Inventors: Masoud M. Kamali, San Francisco, CA (US); Dariush Anooshfar, San Jose, CA (US); Anoosh Abdy, Menlo Park, CA (US); Arash Ahani, San Mateo, CA (US); Arthur Hsi, Belmont, CA (US); Sadri Zahir, Saratoga, CA (US); Yeliz Ustabas, Atherton, CA (US)

(21) Appl. No.: 18/207,336

(22) Filed: Jun. 8, 2023

Related U.S. Application Data
(62) Division of application No. 14/829,592, filed on Aug. 18, 2015.
(60) Provisional application No. 62/041,561, filed on Aug. 25, 2014.

(51) Int. Cl. A61B 5/11; A61B 5/00

(52) U.S. Cl. CPC A61B 5/1123; A61B 5/6801; A61B 5/1118

(57) ABSTRACT

A real-time human activity recognition (rtHAR) engine embedded in a wearable device monitors a user's activities through the wearable device's sensors. The rtHAR uses the signals from the sensors to determine where the wearable device is relative to the user's body, and then determines the type of activity the user engages in depending upon the location of the wearable device relative to the user's body. The rtHAR is preferably installed on the wearable device as an embedded system, such as an operating system library or a module within software installed on the wearable device, so as to improve the quality of direct feedback from the wearable device to the user, and to minimize the amount of data sent from the wearable device to external archival and processing systems.

This application is a divisional of non-provisional application Ser. No. 14/829,592, which claims priority to U.S. provisional application 62/041,561 filed on Jul. 18, 2015. This and all other extrinsic references referenced herein are incorporated by reference in their entirety.

### FIELD OF THE INVENTION

The field of the invention is monitoring devices

### BACKGROUND

The background description includes information that may be useful in understanding the present invention. It is not an admission that any of the information provided herein is prior art or relevant to the presently claimed invention, or that any publication specifically or implicitly referenced is prior art.

All publications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

In today's ever health-conscious world, accurately monitoring a user's activity is of paramount importance in order to analyze health trends and changes. For years, athletes have been tracking their health progress using notebooks and training schedules, and have even been able to manually input their daily workouts into helpful computer applications. Manually entering a user's daily activities into a log, however, is often time-consuming and the extra time it takes to log such data can often dissuade users from keeping a complete log of their activities. Fortunately, automatic sensors can be used in limited ways to help automatically track the activities of users.

US 2014/0028539 to Newham teaches a system that detects a user's real-time hand gestures from wireless sensors that are placed on the user's body. Short-range radio transmissions are sent as the user moves his/her hands, and a computing device tracks the hand positions of the user over time, compares the movements to predefined patterns, and initiates computer commands depending upon the recognized gesture. Newham's system, however, requires the user to be next to a computing device that receives and processes the sensor information in real-time in order to determine the type of gesture the user is making. Many users cannot always ensure that all of their movements are performed within range of a radio-frequency (RF) receiving computing device at all times. Any gestures made by a user outside the range of Newham's computing device are not recorded.

US 2015/0045700 to Cavanagh teaches a patient monitoring system with multiple sensors attached to places on a patient that are proximal to joints of the patient. For example, an accelerometer and a goniometer could be attached to a patient's knee along with a transmitter that wirelessly transmits data acquired from the sensors to a computer. The computer could then recognize a joint flexion movement and determine an extent of movement of the joint between flexion and extension of the joint. Cavanagh, however, also requires the user to be within transmitting range of a computer in order to translate the data received from the sensors mounted on the body.

U.S. Pat. No. 8,903,671 to Park teaches a wearable wristband that has sensors that collect data from the user, such as accelerometers that sense acceleration or gyroscopes that sense rotation data. The sensor data is then converted into activity data. For example acceleration data could be converted into activity metrics such as “steps taken,” “stairs climbed,” or distance traveled.” The activity metrics are saved on the wristband, and the activity metrics could then be uploaded to a server at a later time via a wireless transmitter. Park's wristband, however, will be inaccurate when placed in a backpack or on a user's foot, because the device only recognizes movements made from the wrist or on the user's belt. If the device is moved to another part of the user's body, the readings will be inaccurate.

Thus, there remains a need for a system and method for monitoring a user's activities throughout the day.

### SUMMARY OF THE INVENTION

The following description includes information that may be useful in understanding the present invention. It is not an admission that any of the information provided herein is prior art or relevant to the presently claimed invention, or that any publication specifically or implicitly referenced is prior art.

The inventive subject matter provides apparatus, systems, and methods for providing an activity tracking device that accurately tracks the activities of a user no matter where the device is located relative to the user's body. The activity tracking device could be a wearable device, such as a bracelet, ankle bracelet, necklace, or hat, or could be a device that is coupled to the user in some fashion, such as placed in a user's pocket or purse, or attached via a pin, button, or clasp. Contemplated activity tracking devices include any computer system having a processor, memory, and a set of sensors that detect information about a user. Embodiments of activity tracking devices comprise mobile computer devices such as tablets, mobile phones, and PDAs, as well as smaller, targeted computer devices such as electronic watches, pendants, earrings, anklets, lockets, pocket monitors, and implantable devices.

The activity tracking device generally has one or more embedded sensors that collect user data, such as an accelerometer, gyroscope, thermometer, barometer, magnetometer, altimeter, photo detectors, pressure sensors, heart rate monitors, blood pressure monitors, and cameras. A sensor module is configured to receive sensor inputs from one or more sensors of the device. While the sensor module could be running on a remote computer system that communicates with the activity tracking device via a wired or wireless interface, the sensor module is preferably installed on the activity tracking device itself. In some embodiments, the sensor module could be configured to also receive sensor inputs from sensors that are not embedded in the activity tracking device. For example an activity tracking device worn on the user's wrist could receive sensor inputs from a device worn on the user's ankle and/or hip, or could receive sensor inputs from a remote camera monitoring the user. Preferably, remote devices send raw data, such as vector information, to the activity tracking device, but in some embodiments the sensor inputs from remote devices are processed in some manner by the remote devices to minimize transmission traffic. For example, a remote device worn on the user's ankle could determine that the user has been running at 9 mph for the last 2 seconds, and could transmit that processed data instead of all of the raw sensor vectors to the activity tracking. Data could be transmitted activity tracking device and remote devices via a wired interface, but is preferably transferred using a wireless interface, such as a Wi-Fi transmitter, a Bluetooth™ transmitter, an RF transmitter, or an IR transmitter.

The sensor module is preferably configured to constantly receive sensor inputs from the sensors in real-time. As used herein, “real-time” means that sensor inputs are received by the sensor module at most every 3 seconds, and preferably at most every 2 seconds, 1 second, 0.5 seconds, every 0.1 seconds 0.05 seconds, or even 0.028 seconds. The system could configure the sensor module to regularly poll the sensors for updated information, for example through a function call, or could configure the sensors to regularly transmit updated sensor input data to the sensor module. Generally the sensor module is configured to accumulate sensor inputs over time in order to determine a general trend of movements. For example, the sensor module could accumulate the last second, the last 2 seconds, the last 5 seconds, the last 10 seconds, or even the last 30 seconds of sensor inputs. The sensor module generally saves such accumulated sensor input information in a memory of the system, and could be configured to save hours or even days of raw sensor information to be analyzed by other modules of the system. In some embodiments, the most recent sensor information (e.g. the last 2 seconds of collected sensor information) is periodically analyzed by a body area module that analyzes the sensor inputs to determine where the device is located relative to the user's body, and/or an activity module that analyzes the sensor inputs to determine what type of activity the user is performing.

A body area module of the system is generally configured to automatically select a body area of the user as a function of the sensor inputs. The body area module could be installed on a separate computer system from the activity tracking device, but is preferably installed on a memory of the activity tracking device itself. In some embodiments, the body area module is configured to select the body area by comparing the sensor inputs to a set of known body area movement signatures. In some embodiments, the system is configured to have a body area database containing known body area movement signatures. The system could also have known body area movement signatures that are differentiated by how they are attached to the body. For example, sensor inputs for an activity tracking device that is held in a user's hand might be different than an activity tracking device that is coupled to the user's wrist via a band. Thus, the body area module could determine not only where the activity tracking device is relative to the user's body, but how the activity tracking device is coupled to the user's body as well. Preferably, the body area database is also installed on a memory of the tracking device itself, such that the tracking device can always know where, relative to the user's body, the tracking device is located.

Preferably, the body area module periodically analyzes the sensor inputs to determine if a location of the activity tracking device has changed relative to the user's body, for example at most every 10 seconds, 5 seconds, 2 seconds, 1 second, 0.5 seconds, 0.1 seconds, 0.05 seconds, or even 0.025 seconds. For example, if a user holds the activity tracking device in his/her hands, and then switches the location of the activity tracking device to his/her pocket, the sensor inputs will likely change over time. The body area module could periodically compare the sensor inputs against its set of known body area movement signatures to determine that the sensor inputs that used to match known body area movement signature of a user's hand have not changed to be similar to known body area movement signatures of a user's pocket. The body area module is preferably configured to automatically transmit the selected body area to the activity module of the system.

An activity module of the system is generally configured to select an activity of the user as a function of the sensor inputs and the body area of the user automatically selected by the body area module. The activity module could also be installed on a separate computer system from the activity tracking device, but is also preferably installed on a memory of the activity tracking device itself. In some embodiments, the activity module is configured to select the activity by comparing the sensor inputs to a set of known activity signatures corresponding with the body area selected by the body area module. In some embodiments, the system is configured to have an activity database containing known activity signatures corresponding to various areas of the body and/or corresponding to the manner in which the activity tracking device is coupled to the user's body. Known activity movement signatures could comprise, for example, signatures for a wrist of the body (held in the hand or coupled to the wrist), a forearm of the body, a bicep of the body, a pocket of the body, a backpack of the body, a shoe of the body, an ankle of the body, a belt of the body, a necklace of the body, a collar of the body, or a hat of the body. Preferably, the activity database is also installed on a memory of the tracking device itself, such that the tracking device can always what type of activity the user is engaged in. While the activity database generally holds activity signatures for a variety of locations of the body (and, in some embodiments, a variety of attachment mechanisms for the activity tracking device), the activity module preferably only compares the sensor inputs against signatures that correspond with the selected body area of the user (and possibly the selected manner in which the activity tracking module is coupled to the body of the user).

Preferably, the activity module periodically analyzes the sensor inputs to determine if a location of the activity tracking device has changed relative to the user's body, for example at most every 10 seconds, 5 seconds, 2 seconds, 1 second, 0.5 seconds, 0.1 seconds, 0.05 seconds, or even 0.025 seconds. The activity module preferably has an extensive list of activity signatures to compare the sensor inputs to, in order to determine different types of body activities. For example, the known activity signatures could comprise activity signatures for running, walking, being motionless, sleeping, resting, changing elevation, turning, swimming, and riding in a vehicle. The activity module is preferably configured to transmit the currently detected activity to an interface module configured to present the selected activity to an interface of the wearable device. In some embodiments, the interface of the wearable device could be a display of the wearable device. Preferably, the interface presents a combination of the selected activity, and some selected raw sensor information. For example, the interface could present that the user is walking at 5.0 miles per hour at a first time, then changed to jogging at 5.0 miles per hour at a second time, then changed to running at 8.0 miles per hour at a third time, then changed to walking at 4.0 miles per hour at a fourth time. Other processed data, such as the rate of acceleration/deceleration and the amount of torque applied to the user's core during each step movement could also be presented to the interface of the activity tracking device.

While each of the aforementioned modules—the body area module, the activity module, and the interface module—could be installed on a separate, remote device that communicates with the activity tracking device through a wired or wireless interface, the modules are all preferably installed on a memory of the activity tracking device so as to be all self-contained within a single embedded system. Preferably, the modules are provided as a software library, such as an SDK, that programmers of the activity tracking device could utilize to gain specific information regarding how the user is moving. In other embodiments, the modules could be embedded in a software application that is installed on the activity tracking device.

Various objects, features, aspects and advantages of the inventive subject matter will become more apparent from the following detailed description of preferred embodiments, along with the accompanying drawing figures in which like numerals represent like components.

The following discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

### BRIEF DESCRIPTION OF THE DRAWING

FIG. 1 is a hardware schematic of a system of the current invention.

FIG. 2 is a software schematic of an embodiment of an activity tracking device.

FIG. 3 is a flowchart of steps taken by an inventive system to track activity of a user.

FIG. 4 shows an embodiment of a state machine of an activity tracking device.

FIG. 5 is another software schematic of an embodiment of an activity tracking device.

FIG. 6 is a software schematic of an embodiment of a mobile phone modified to act as an activity tracking device.

FIG. 7 is a software schematic of an embodiment of a mobile phone modified to act as a sleep tracking activity tracking device.

FIG. 8 is a software schematic of an embodiment of an activity tracking device without an operating system.

FIG. 9 is a software schematic of an embodiment of an activity tracking device having separate device adaptation layers.

FIG. 10 is a software schematic of an embodiment of an activity tracking device within a distributed system.

FIG. 11 is a software schematic of an alternative sleep tracking activity tracking device that uses a motion recognition engine as a service.

FIG. 12 is a software schematic of an alternative activity tracking device having an application that uses a motion recognition engine as an embedded service.

FIG. 13 shows a distributed activity tracking device system.

FIG. 14 shows another distributed activity tracking device system.

FIG. 15 shows an embodiment of an activity tracking device used by a user.

FIG. 16 shows an embodiment of the activity tracking device of FIG. 17 used in a different manner by the same user.

FIG. 17 shows a user interface to provide a body area signature to an activity tracking device.

FIG. 18 shows a user interface that displays a log of activity detail records.

### DETAILED DESCRIPTION

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously. For example, a watch that is wrapped around a user's wrist is directly coupled to the user, whereas a phone that is placed in a backpack or a pocket of a user, or a pin that is pinned to the lapel of a user's shirt, is indirectly coupled to the user. Electronic computer devices that are logically coupled to one another could be coupled together using either wired or wireless connections in a manner that allows data to be transmitted from one electronic computer device or another electronic computer device, and may not be physically coupled to one another.

Unless the context dictates the contrary, all ranges set forth herein should be interpreted as being inclusive of their endpoints, and open-ended ranges should be interpreted to include commercially practical values. Similarly, all lists of values should be considered as inclusive of intermediate values unless the context indicates the contrary.

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

It should be noted that any language directed to a computer device or computer system should be read to include any suitable combination of computing devices, including servers, interfaces, systems, databases, agents, peers, engines, controllers, or other types of computing devices operating individually or collectively. One should appreciate the computing devices comprise a processor configured to execute software instructions stored on a tangible, non-transitory computer readable storage medium (e.g., hard drive, solid state drive, RAM, flash, ROM, etc.). The software instructions preferably configure the computing device to provide the roles, responsibilities, or other functionality as discussed below with respect to the disclosed apparatus. In especially preferred embodiments, the various servers, systems, databases, or interfaces exchange data using standardized protocols or algorithms, possibly based on HTTP, HTTPS, AES, public-private key exchanges, web service APIs, known financial transaction protocols, or other electronic information exchanging methods. Data exchanges preferably are conducted over a packet-switched network, the Internet, LAN, WAN, VPN, or other type of packet switched network.

One should appreciate that the disclosed techniques provide many advantageous technical effects including the ability to track the details of a user's activities using an activity tracker device that could be directly or indirectly coupled to the user in a variety of ways, without needing to specially configure or alter the activity tracker device.

The inventive subject matter provides apparatus, systems, and methods in which an activity tracker device dynamically tracks the activities of a user no matter where the activity tracker device is in relationship to a user.

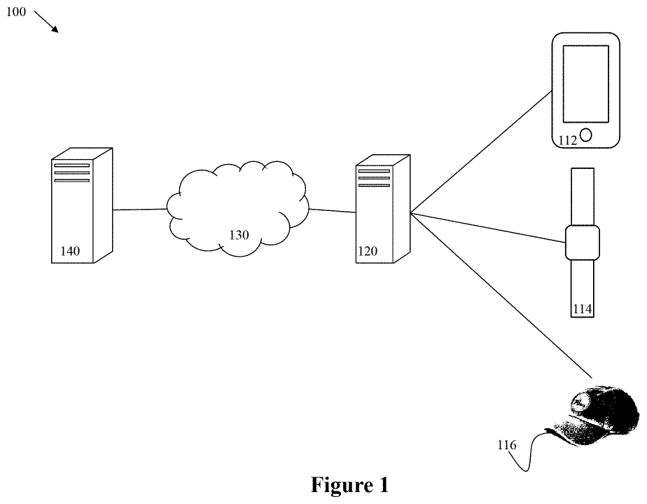

In FIG. 1, a system 100 has a plurality of activity tracker devices 112, 114, and 116 that communicate processed, logged data to server 120, which is logically coupled to a remote server 140 through network 130. Each of activity tracker devices 112, 114, and 116 comprises a computer system having a processor, a memory, a plurality of electronic sensors, and software saved on the memory that, when executed by the processor, gleans data from the plurality of electronic sensors in order to collect activity data from a user of each of activity tracker devices 112, 114, and 116. While activity tracker device 112 is shown as a mobile phone, activity tracker device 114 is shown as a watch, and activity tracker device 116 is shown as a hat, each having a plurality of sensors that are read by the system, the activity tracker devices could be any computer system having one or more sensors that could be used by the system to track information about a user's movements. Preferably, each activity tracker device works independently of the other activity tracker device, and tracks the movement of any user (not shown) using the activity tracker device based upon information detected by the sensors.

Each activity tracker device translates the sensor information into a log of activity detail records (ADR), which is a log of activities over time that the activity tracker device has detected through the device's sensors. When any of the activity tracker devices 112, 114, or 116 are logically connected to server 120, that information could be transmitted to server 120 for further analysis. For example, server 120 might hold an ADR log for a user over days, weeks, months, or even years, and could be configured to analyze trends in the user's daily activities. Such information could also be saved into the cloud or aggregated among a plurality of users by transmitting the data to distal server 140 through network 130.

Activity tracker devices 112, 114, and/or 116 could be logically connected to server 120 in any suitable manner, for example through a wired USB, Ethernet, or serial connection, or through a wireless WiFi, Bluetooth, infrared, satellite, or cellular phone connection. Network 130 could be any network, such as a WAN, LAN, or the Internet, and generally connects to distal servers that could act as a backup storage repository for a single user. While servers 120 and 140 are shown as desktop computer systems, server 120 and/or server 140 could be any computer system, such as a mobile phone device, a laptop, a tablet, a server blade, or a server cluster without departing from the scope of the current invention.

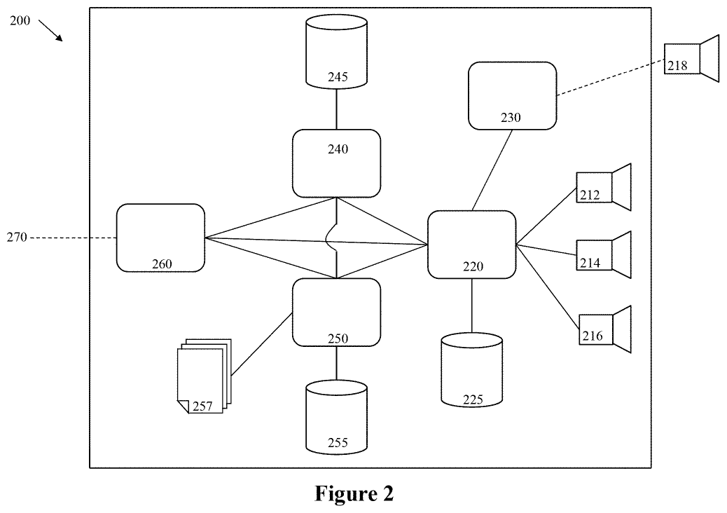

In FIG. 2, a software schematic 200 of an activity tracking device has embedded sensors 212, 214, and 216 monitored with sensor module 220, distal sensor 218 monitored with sensor module 220 via external interface 230, body area module 240 that analyzes data collected by sensor module 220, activity module 250 that analyzes data collected by sensor module 220 and body module 240, and interface module 260 that collects data from at least one of activity module 250, body area module 240, and sensor module 220 to be transmitted to an internal interface 270 of activity tracking device. Embedded sensors 212, 214, and 216 are sensors that are embedded in the activity tracking device itself, while distal sensor 218 is a sensor that is located on a distal device, separate from the activity tracking device. Contemplated sensors include accelerometers, gyroscopes, thermometers, barometers, magnetometers, altimeters, photo detectors, pressure sensors, heart rate monitors, blood pressure monitors, and cameras. Raw data from the sensors are received by sensor module 220, either directly through a function call to the sensor itself, or indirectly through external interface 230. External interface 230 is an electronic interface that is configured to retrieve sensor data from a distal sensor and transmit the sensor data to sensor module 220.

Sensor module 220 is preferably installed on the activity tracking device memory itself, such as activity tracking device 112 or activity tracking device 114, although sensor module 220 could alternatively or additionally be installed on server 120 without departing from the scope of the current invention. Contemplated sensor data includes any values that a sensor could provide, for example numerical values for a temperature or a barometer, vectors for accelerometers or gyroscopes, or digitized representations for microphones and cameras. Sensor module 220 could be configured to archive the raw sensor data to sensor input database 225, which is a database of memory that holds archived sensor data over time. In some embodiments, sensor input database 225 only holds enough archived sensor information to be relevant to body area module 240 or activity module 250, such as only the last 30 minutes, the last 20 minutes, the last 10 minutes, the last 5 minutes, the last 2 minutes, the last minute, the last 30 seconds, 20 seconds, 10 seconds, 5 seconds, 2 seconds, 1 second, or 0.5 seconds of collected sensor data. The system could be customized to have frames of time that are as long or as short as the programmer wishes, from 0.28 milliseconds to 30 minutes. In such embodiments, database 225 is preferably configured to be some sort of circular buffer that periodically overwrites historical data over time in order to conserve space. In other embodiments, sensor input database 225 is configured to hold much larger amounts of data from the provided sensors until the log of archived sensor data can be offloaded to a separate computer system, such as server 120. For example, sensor input database 225 could be configured to hold as much as a day's, a week's, or even a month's worth of sensor data if need be.

Body area module 240 is configured to retrieve sensor data from sensor module 220, and determine the context of how the user is utilizing the activity tracking device. Body area module 240 could do this by comparing the latest sensor input data against body area signatures stored in body area signature database 245. Body area signature database 245 is typically a database of signatures stored in memory which give an indication as to the user's context as related to the activity tracking device. For example, when a user is carrying the activity tracking device in the user's pocket, the received sensor data could have one type of signature, whereas when a user has the activity tracking device clipped onto a belt, the received sensor data could have another type of signature. Signatures do not have to be limited to the position of the activity tracking device's location relative to the user's body, but could provide any suitable context, for example how the activity tracking device is coupled to the user's body, whether the user is riding in a vehicle, how high the user presently is, the present speed of the user, and/or the location of the user (e.g. GPS coordinates). Preferably, body area module 240 constantly evaluates received sensor input data periodically over time so as to keep the system's knowledge of the activity tracking device's context current. Body area module 240 is preferably configured to compare the sensor input data against various signatures in database 245, and select a body area context as a function of the sensor input data as compared with the various signatures in database 245.

Contemplated body area contexts include signatures for a wrist of the body (held in the hand or coupled to the wrist), a forearm of the body, a bicep of the body, a pocket of the body, a backpack of the body, a shoe of the body, an ankle of the body, a belt of the body, a necklace of the body, a collar of the body, or a hat of the body. Body area contexts do not need to be limited to areas of the body, and could include additional contextual signature information, such as whether the activity tracking device is stuck to the user's skin, is attached to the user via a band, is coupled to a non-human user (e.g. a dog, a cat, or an inanimate object), whether the user is in a car, plane, or train, whether the user is indoors or outdoors, whether the user is resting or sleeping, and/or whether the user has any infirmities. Body area contexts are particularly useful for inanimate objects, for example packages, that are transported from one location to another. Once the package is picked up, the system could detect where, in relation to the user, the package is, how it is being handled, whether the user drops the package, etc. Each activity from shipping to delivery could then be tracked in detail, even while a user holds the package with his/her hands, rests it on his/her shoulder, or places it on a dolly to transport to another location. Some body area context signatures may require a longer aggregate of sensor input than other context signatures, for example one body area context signature may require a sample input aggregate size of 3 minutes while another body area context signature only requires a sample input aggregate size of 10 seconds. Some body area context signatures may be selected as a function of past selected body area contexts and/or past selected activities, for example a body area context of the user sleeping may only be detected if the user is detected to be engaged in the resting activity for more than 10 minutes. Other body area context signatures may be selected as a function of other sensors, such as time (only detect a sleep context after certain hours) or location (only detect a sports context when the user is within a few hundred feet of a gymnasium).

In some embodiments, a user might have user-specific body area signatures that are not known by the system, or may be particular to a specific user. For example, the user might have different contexts than other users, and might need a customized context, such as for a user that is extremely short or a user that performs rare contextual activities. For example, a user could provide signatures to body area database 245, either by importing them directly to body area database 245, or by indicating to the system that the user is using the activity tracking device in a particular manner, and has the system “record” the sensor input data into body area signature database 245 as a signature for that particular context. For example, the user could move a certain way while attaching the activity tracking device to the user's outer thigh in a gun holster, and could indicate to the system through a user interface (via internal interface 270) that the user is attaching the activity tracking device in a new body area context, could hit “record” and have sensor data customized for the particular user be recorded into body area signature database 245 over time as a signature for that new body area context. In some embodiments, the user could lock the signature saved in the database to a unique identifier (UID) of the user, so that the system only compares sensor inputs against the user-specific body area context signatures when the system first authenticates that user and associates use of the activity tracking module with the UID.

In some embodiments, body area module 240 could be trained to prefer one context over another by a user. For example, where body area module 240 detects that the user's context is similar to two different body area context signatures (e.g. the module detects that the sensor inputs are similar to the device being placed in the user's pocket or the device being placed in the user's backpack), the module could transmit an alert to the user via a user interface (e.g. via internal interface 270) and allow the user to choose to prefer one context over another context by selecting one of the two. As used herein, a module that determines that sensor input data is similar to two or more different contexts is one that compares the sensor input data to all known body area context signatures, ranks the body area context signatures according to similarity, and determines that the top ranked body area context signatures are within a 3%, 2%, 1%, or even 0.5% similarity with one another.

In other embodiments, body area module 240 might select several body area contexts and send a plurality of the body area contexts to activity monitor 250. In such an embodiment, activity monitor 250 would track the user's activities over a plurality of contexts (e.g. track the user's activity as if the activity tracking device were coupled to the user's head or the user's hip). Over time, body area module 240 will select a preference of one body area context over another (e.g. will detect a higher similarity between the sensor inputs and the body area context signature for a user's head than the user's hip), and will delete the user activity log for the less preferred body area context. Thus, the activity tracking module could track activities across a plurality of contexts when confusion arises (i.e. determines that sensor input data is similar to two or more different contexts), and resolve that confusion at a later time.

Activity module 250 is configured to receive one or more body area contexts from body area module 240, and select a user activity as a function of the sensor inputs as they relate to the selected body area context. Activity module 250 has activity database 255, which contains activity signatures for several activities that the user could engage in. The activity signatures are generally associated with a body area context. Preferably, activity module 250 only compares the input sensor data against the body area context for activity signatures that correspond with the selected body area context(s). Contemplated activities include walking, running, resting, sitting, driving, flying, playing specific sports (e.g. volleyball, basketball, tennis, soccer), jumping, sitting, falling, squatting, skipping, sprinting, punching, kicking, or doing yoga. The set of potential activities could also change from context to context. For example, the set of potential activities to be selected for a user at rest could comprise reading, watching TV, playing a game, or getting a massage, while the set of potential activities to be selected for a user that is asleep could comprise restless sleep, restful sleep, deep sleep, silent sleep, and snoring sleep. Activities detected for a device that is coupled to a dog would be different than activities detected for a device that is coupled to a human. Thus, when body area module 240 selects a dog context or a sleeping context for the user, different sets of activity signatures would be selected from activity database 255.

The selected activity is preferably archived and saved in a log 257 of activity detail records for the user. The log could be detailed, containing granular data of activities, contexts, and/or sensor input data for every few seconds or even milliseconds of time, but is preferably filtered to convey summarized information, such as time stamps of when the user's activity changed from one activity to the next, and a summary of the activity. For example, where activity module 250 detects that a user walked, then ran, then rested, the speed at which the user walked and the speed at which the user ran might vary considerably over time, but activity module 250 might simply summarize the log information as a first time period labeled “walking” with a first average speed, a second time period labeled “running” with a second average speed, and a third time period labeled “resting.”

The system could then present any of the logged data to an internal interface 270 of the activity tracking device via interface module 260. Internal interface 270 could be any suitable interface of the activity tracking device, such as an interface to a display of the tracking device to display the current detected activity (or a portion of the log of the ADR), an interface to a speaker of the tracking device to verbalize the current detected activity, or an interface to an application of the activity tracking device. In some embodiments, the system might not keep a log 257 of the ADR records of the user, and instead might simply continuously output the current activity to internal interface 270. An application that receives the current activity from interface module 260 via internal interface 270 could then construct its own log of the ADR and parse the data accordingly. Interface module 260 could be configured to present contextual data to internal interface 270 as well. This is particularly useful where body area module 240 detects a dramatic change in body area context, such as when the user attaches or detaches the activity tracking module to a part of the body. For example, the activity tracking module might be set to go to a power-saving sleep mode when the user detaches the activity tracking module from a part of the body.

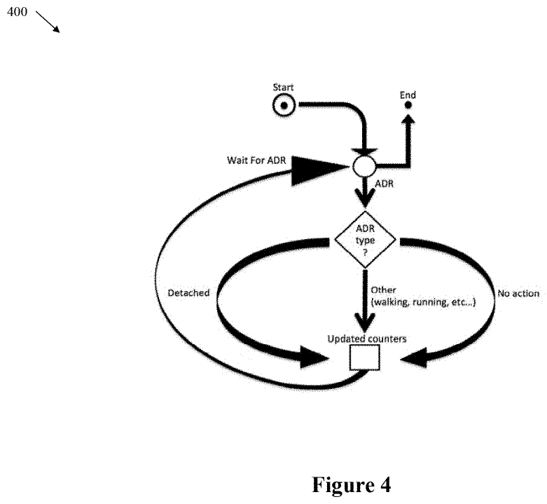

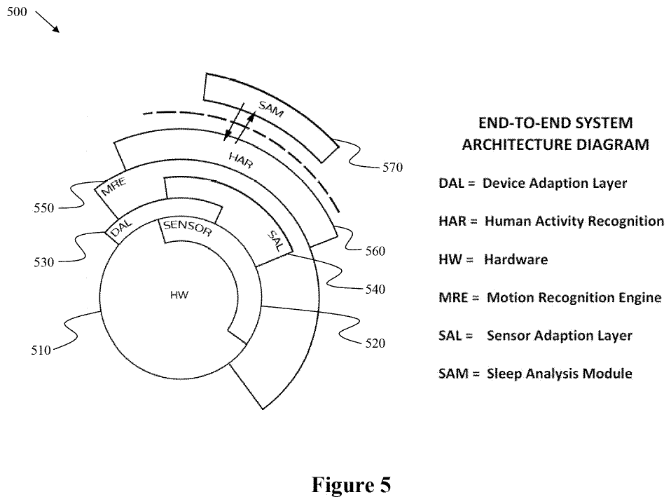

While each of sensor module 220, external interface 230, body area module 240, activity module 250, and interface module 260 are preferably installed on the activity tracking device itself, for example via an application on the device, an SDK on the device, or as a part of the operating system of the device, each of the modules could be installed on other systems, such as server 120 or even server 140, without departing from the scope of the invention. Modules 220, 240, 250, and 260 are preferably embedded in the activity tracking device to dynamically track a user's activities in real-time. Alternative software architectures will be discussed in FIGS. 5-10.

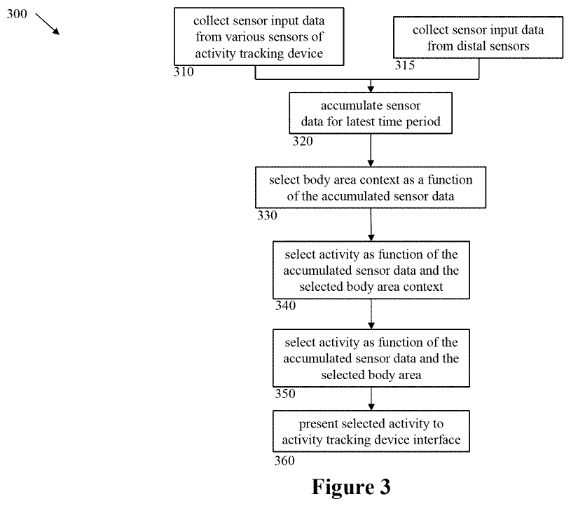

FIG. 3 is a flowchart 300 of various steps that could be taken by an inventive system to track activity of a user. In steps 310 and, optionally, step 315, the system collects sensor input data from various sensors. The collected sensor input data is accumulated by the system in step 320 for the latest time period. In some embodiments, the system could accumulate all sensor input data, but preferably the system uses a circular buffer that only saves accumulated sensor input data for the latest time period. In step 330 the system selects the body area context as a function of the accumulated sensor data, and in step 340 the system selects a detected activity of the user as a function of the accumulated sensor data and the selected body area context. The system then presents the selected activity to an activity tracking device interface in step 360.

FIG. 4 shows a simple state machine 400 for an embodiment of an activity tracking device that accepts activity log information from an interface module. In state machine 400, once the state machine starts, the state machine receives a single activity (ADR) from the interface module. The state machine then determines what type of activity is detected from the activity tracking device. A “detached” activity means that the system has detected that the activity tracking device is detached from the user and is not tracking any activities from the user. A “no action” activity means that the system has detected that the user has not taken any actions, or has taken an action that has not been defined by the system. An “other” activity means that the system has detected a specific activity from the user that matches one of the known activity signatures, such as running, walking, jumping, sitting, etc. The state machine then increments the counter for the detected activity, which logs a time-stamp for the changed activity. Then the state machine returns back to the top empty circle to wait for a new ADR different from the old ADR. As time passes, the counters compose a log of ADR information for the user, until the system is deactivated at the “end” state. Such a state machine is very simplistic in its function, but demonstrates the power of this system to dynamically detect a user's activity no matter what the context of the activity tracking device.

FIG. 5 shows a simplified software schematic 500 of the present invention, showing how different modules might be coupled with one another. Here, the hardware architecture 510 represents the underlying hardware architecture of the activity tracking device, such as the device's system BUS, or the device's system operating system. Sensor architecture 520 represents the software architecture of the activity tracker device's sensors. Hardware architecture 510 and sensor architecture 520 generally varies from device to device, such as a Samsung™ galaxy phone vs. an iPhone™ vs. a Pebble™ steel. In preferred systems, a device adaptation layer 530 and a sensor adaptation layer 540 are layered on top of the hardware architecture 510 and the sensor architecture 520 in order to provide a common basis for the motion recognition engine 550 to interact with. In some systems, device adaptation layer 530 and sensor adaptation layer 540 are built to be device-specific and sensor-specific, respectively, so as to leave a small footprint. In other systems, device adaptation layer 530 and sensor adaptation layer 540 are built to be able to communicate with a plurality of devices and/or a plurality of sensors in order to create a software package that is hardware agnostic.

In this manner, a common motion recognition engine 550 could be applied to any hardware having any type of sensor. Motion recognition engine 550 is generally configured to read the raw sensor input data and translate the raw sensor input data into motions that are easier to parse. For example, motion recognition engine 550 could read a vector from an accelerometer over time and translate that vector into an acceleration jerk to a speed of 10 mph over 2 seconds, could read a pressure drop from a barometer over time and translate that pressure drop into a gain in altitude, or could read a body turning 25 degrees left during a run. While motion recognition engine 550 preferably only interacts with the activity tracking device via sensor adaptation layer 540 or device adaptation layer 530, in some embodiments motion recognition engine 550 could be configured to also directly interact with hardware architecture 510 and/or sensor architecture 520. This is particularly useful when there are hardware-specific updates that need to be quickly applied to motion recognition engine 550.

In some embodiments, motion recognition engine 550 might be installed on one hardware architecture with one set of sensors, and on a different hardware architecture with a different set of sensors. Preferably, motion recognition engine 550 is configured to have signatures that could be applied to different sets of sensors. For example, where a first hardware architecture has only an accelerometer and a gyroscope, and a second hardware architecture has an accelerometer, gyroscope, and a barometer, motion recognition engine 550 might use more nuanced signatures (signatures for sensor inputs of an accelerometer, gyroscope and barometer) for the second hardware architecture than for the first hardware architecture (signatures for sensor inputs for just an accelerometer and a gyroscope).

Motion recognition engine 550 provides an interface to human activity recognition engine 560, which recognizes the activity of the user based upon the recognized activity of the user—as applied to the recognized body area context of the user. For example, while motion recognition engine 550 might recognize a motion as an acceleration from 2 mph to 8 mph, human activity recognition engine 560 might recognize a start of a sprint. Human activity recognition engine 560 recognizes one or more specific activities, and transmits them to application 570. Here, application 570 represents a sleep analysis module, which analyzes detected human activities from the human activity recognition engine 560, and tracks the sleep accordingly. Application 570 might be configured to analyze the activities reported by human activity recognition engine 560 and determine additional contexts, which are then fed back to human activity recognition engine 560 to help modify the types of contexts detected. For example, application 570 might detect that the user has had over 2 hours of non-REM sleep based upon detected activities by the human activity recognition engine 560. This information could be fed to human activity recognition engine 560, which would then modify the detected context from “asleep with an activity tracking device coupled to the wrist” to “interrupted sleep with an activity tracking device coupled to the wrist,” triggering a modified set of activity signatures correlating to the new context of “interrupted sleep with an activity tracking device coupled to the wrist.”

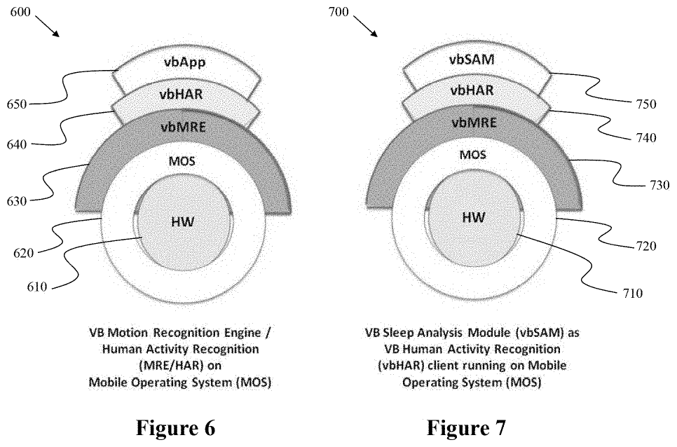

Such modified architectures could be applied on various system architectures as well. FIG. 6 shows a software architecture 600 of the inventive system as applied to a mobile operating system. The hardware architecture 610 of the mobile device is completely enveloped by mobile operating system 620, preventing the software from running machine code on the device at all. A motion recognition engine 630 is layered on top of mobile operating system 620 to poll the sensors of the mobile operating system and determine what types of motions can be detected by analyzing sensor inputs. Human activity recognition engine 640 is layered on top of motion recognition engine 630 to detect different types of activities of the user, and an application 650 is then layered on top of the human activity recognition engine 640.

In this manner, a generic human activity recognition engine 640 could be layered on a specific motion recognition engine 630 designed specifically to communicate with the mobile operating system 620. Any application 650 designed to read and interpret activities detected by the generic human activity recognition engine 640 could then be installed on a plurality of different mobile operating systems. FIG. 7 shows similar software architecture 700, having hardware 710, mobile operating system 720, specific motion recognition engine 730 and generic human activity recognition engine 740. However, in software architecture 700, a specific type of application, a sleep analysis module 750, is installed on the system. In this manner, the sleep analysis module 750 could be installed on a plurality of different types of mobile operating systems without having to customize the software for differing mobile operating systems.

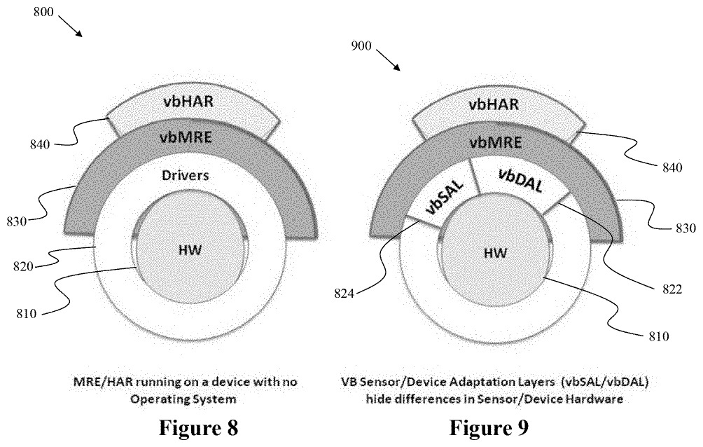

FIG. 8 shows a software schematic 800 of a motion recognition engine mounted to a device that fails to have any kind of operating system at all. In software schematic 800, the device's hardware architecture 810 is only accessible via device drivers 820. Device drivers 820 are able to poll the sensors of hardware architecture 810 for any software that is written specifically to poll sensor information without use of an operating system. Motion recognition engine 830 is typically written specifically for use with systems having no operating system, only containing function calls to drivers 820. Motion recognition engine 830 polls drivers 820 for sensor input information, and translates that sensor input into motions recognizable by generic human activity recognition engine 840. In this way, any generic human activity recognition engine 840 could be installed on an activity tracking device without an operating system, allowing any third party to plug into the human activity recognition engine 840 in a device-agnostic manner.

FIG. 9 shows a software schematic 900 of motion recognition engine mounted to a device that fails to have any kind of device drivers at all. Here, the hardware architecture 810 cannot be accessed by known device drivers, so a device adaptation layer 822 and a sensor adaptation layer 824 must be installed, similar to software schematic 500. Device adaptation layer 822 is preferably configured to be able to adapt to a plurality of devices and sensor adaptation layer 824 is preferably configured to be able to adapt to a plurality of sensors, although device-specific device adaptation layers and sensor-specific sensor adaptation layers are contemplated. Motion recognition engine 830 could then be installed on top of device adaptation layer 822 and sensor adaptation layer 824 to poll sensor input data and recognize certain motions. A generic human activity recognition engine 840 is then installed on top of motion recognition engine 830 to provide a device-agnostic plug-in to a device that does not have any operating system nor device drivers.

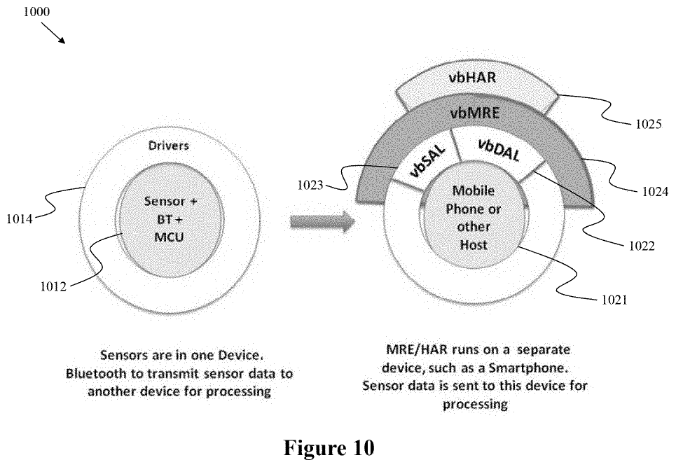

In FIG. 10, a software schematic 1000 has a distributed system of a remote distal sensor that is separate from the activity tracking device. Here, the distal system has a hardware architecture 1012 having a sensor, Bluetooth device, and a micro control unit, such as an ASIC device. Drivers 1014 are installed on hardware architecture 1012 with the sole purpose being to transmit sensor input data to the hardware architecture 1021 of the activity tracking device. Generally, the activity tracking device also has other sensors that provide sensor input data. Device adaptation layer 1022 is configured to communicate with the hardware architecture 1021 of the activity tracking device while sensor adaptation layer 1023 is configured to poll sensor input data both from hardware architecture 1021 of the activity tracking device and hardware architecture 1012 of the distal device. As before, motion recognition engine 1024 is configured to recognize motions from the various sensor inputs, and a generic human activity recognition engine 1025 is installed on motion recognition engine 1024. Motion recognition engine 1024 is configured to read distal sensor inputs and compare them to signature information for a plurality of different body area context signatures to provide even more nuanced data. This is particularly useful, for example, for applications that analyze how different limbs on a user's body work together in an athletic motion. This architecture allows a customized underlying motion recognition engine 1024 to synchronize data from among many different distal systems to a single generic human activity recognition engine 1025.

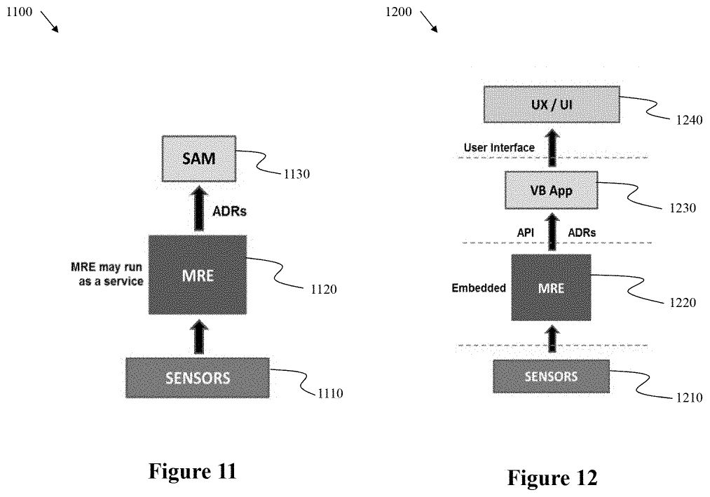

In FIG. 11, a software architecture 1100 shows an embodiment of a sleep tracking activity tracking device that uses a motion recognition engine as a service. In this embodiment, sensors 1110 provide sensor input data to motion recognition engine 1120, which is seen as a black box to any outside applications. The outside applications, such as sleep activity module 1030, do not see any of the raw sensor input data, and instead only monitor a log of recognized activity detail records as they are recognized by the motion recognition engine 1120. Sleep activity module 1030 logs detailed sleep data for the user over time, saves that information to the local memory of the activity tracking device, and exports that data at a later time when needed. There is no need for a distal system to review the raw sensor input data and analyze a user's sleep patterns.

In FIG. 12, an alternative software architecture 1200 shows an embodiment of an application that provides a user interface to the activity tracking device. Here, sensors 1210 provide sensor input data to motion recognition engine 1220, which, again, is seen as a black box to any outside applications. Here, application 1230 monitors a log of recognized activity detail records as they are recognized by the motion recognition engine 1220, and provides a user interface 1240 to a user of the activity tracking device. The user interface 1240 is generally on the activity tracking device itself, such as a screen of a mobile phone or smart watch.

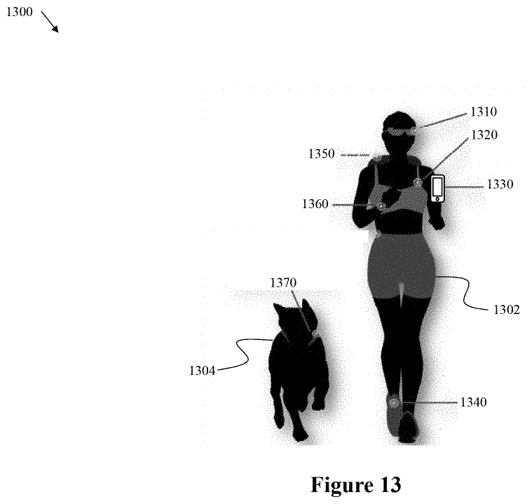

FIG. 13 shows a distributed activity tracking device system 1300 as applied to both a human user 1302 and a non-human user 1304. Human user 1302 has a plurality of sensors 1310, 1320, 1340, 1350, and 1360, and non-human user 1304 has a sensor 1370. Each of the aforementioned sensors are logically coupled to activity tracking device 1330, shown here euphemistically as a mobile phone. Activity tracking device 1330 collects sensor data from each of the sensors to create a nuanced, detailed activity report. The various sensors on human user 1302 could report detailed information on how well the human user 1302 is running. By synchronizing data from so many sensors, activity tracking device 1330 can not only report, with specificity, when human user 1302 started running, walking, and human user 1302's average pace, but activity tracking device 1330 could also report which foot human user 1302 is leading with, how much stress is being applied to different parts of the body, etc. The same underlying software structure could be installed on each of sensors 1310, 1320, 1340, 1350, 1360, and 1370, creating a hardware agnostic distributed environment with a single application that does not need to parse out detailed sensor data from a plurality of different sources. The application running on activity tracking device 1330 needs only to parse out the log of ADR information from each sensor device.

Sensor 1370 transmits recognized motion activity information from non-human user 1304, whose context is recognized because a non-human user 1304 has a different body area contextual signature than human user 1302.

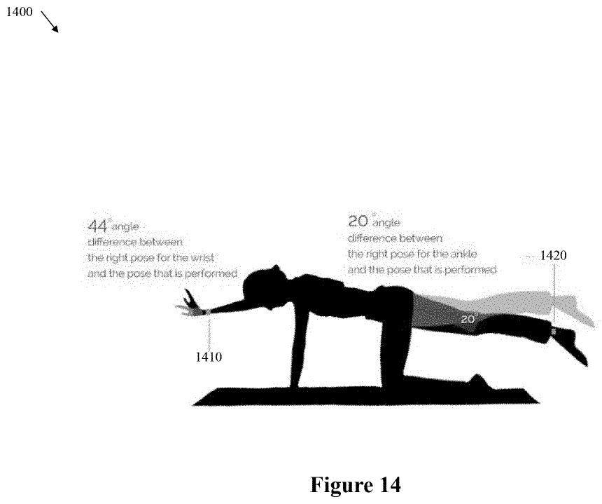

FIG. 14 shows another distributed activity tracking device system 1400 having a first activity tracking device 1410 and a second activity tracking device 1420. As the user moves her arm and leg up and down in the various exercise positions, the different sensors report differing recognized activities. Activity tracking device 1410 recognizes an arm motion having a 44 degree angle difference between the right, recommended pose for the wrist and the actual detected pose that is performed, and activity tracking device 1420 recognizes a leg motion having a 20 degree angle difference between the right, recommended pose for the ankle and the actual detected pose that is performed. Here, the different activity tracking devices could be configured to work on concert together with a single application that transmits a correct, recommended pose signature to each activity tracking device, which provides feedback to the user, such as an auditory sound or a flashing light.

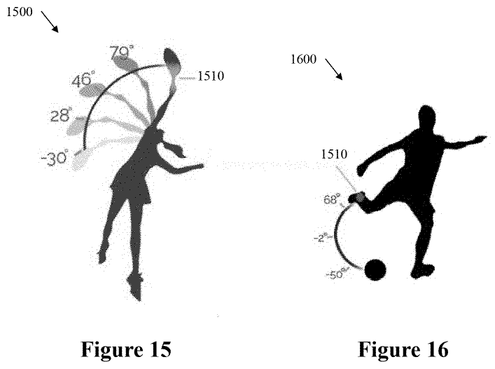

FIGS. 15 and 16 shows an embodiment of an activity tracking device 1510 used by a user 1500 and a user 1600. User 1500 uses the activity tracking device 1510 to track and detect a swinging motion for tennis, while user 1600 uses the activity tracking device 1510 to track and detect a kicking motion for soccer. The same device is used to detect very different motions on different parts of the body without any need to use different devices, since the device automatically detects the body area context that it is used in (the wrist for user 1500 and the ankle for user 1600) and applies the appropriate activity signatures accordingly.

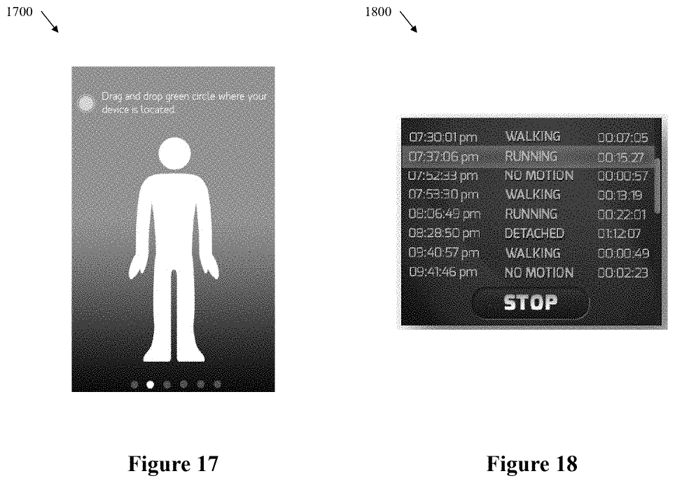

FIG. 17 shows a user interface 1700 that allows a user to provide a body area signature to an activity tracking device. Here, the user has placed the activity tracking device on his/her body, and the system has not recognized the body area signature. The user could then drag and drop the green circle to the area of the body that the activity tracking device has been coupled to, and could add contextual information (e.g. directly coupled to the neck via a necklace, worn underneath a shirt) that could be used by the system in correlation with the body area signature being created. In this manner, a user could add contextual signatures to the body area signature database on the fly through a user interface.

FIG. 18 shows a user interface 1800 that displays a log of activity detail records (ADRs) over periods of time. The log shows time stamps of when the detected activity was detected, and the length of time for each activity. While the total amount of sensor input data sensed by the activity tracking device could be large, the transmitted log of recognized activity is minimal, and provides a very small footprint of data to be transmitted from the activity tracking device. Each activity is depicted as a single “block” of activity, defined as an activity sustained over a period of time.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. A system for assisting a user to reduce stress on the user's body, comprising:
a first activity tracking device, configured to be worn about the user's body at a first body area, and having a first memory configured to store first sensor data from a first movement sensor;
a second activity tracking device, configured to be worn about the user's body at a second body area distant from the first body area, and having a second memory configured to store second sensor data from a second movement sensor;
a computing system configured to receive the first and second sensor date from the first and second activity tracking devices, respectively, and utilize the received first and second sensor data to (a) determine first and second locations of the first and second activity tracking devices, respectively, (b) correlate the received first and second sensor data with different first and second activity signatures respectively, and (c) select an activity of the user based upon the first and second signatures; and
a communication component configured to provide an instruction to the user to reduce the stress by altering an action of the user during performance of the selected activity.

2. The system of claim 1, wherein the sensor module is further configured to receive a portion of the sensor inputs from a second set of sensors on a remote device.

3. The system of claim 1, wherein the computing system is configured to receive at least some of the received first sensor data via a wireless interface.

4. The system of claim 1, wherein the computing system is configured to process the received first sensor data with a granularity of tens of milliseconds.

5. The system of claim 1, wherein the first activity tracking device is configured to accumulate at least 2 seconds of the first sensor data.

6. The system of claim 1, wherein the sensor module is further configured to accumulate up to the last 2 seconds of sensor inputs for use by the activity module.

7. The system of claim 1, wherein the first activity tracking device is configured to accumulate at least 10 seconds of the first sensor data.

8. The system of claim 1, wherein the first movement sensor is selected from an accelerometer and a gyroscope.

9. The system of claim 8, wherein the first activity tracking device includes an auxiliary sensor selected from the list consisting of a thermometer, a barometer, and a magnetometer, and is configured to provide auxiliary sensor data from the auxiliary sensor to the computing system.

10. The system of claim 1, wherein the body area module is further configured to select the body area context by comparing the sensor inputs to a set of known body area movement signatures.

11. The system of claim 10, wherein the body area module and the set of known body area movement signatures are both saved on a memory of the activity tracking device.

12. The system of claim 1, wherein each of the first and second activity signatures are selected from a set of known activity signatures for a wrist, a head, a pocket, a backpack, and a shoe.

13. The system of claim 12, wherein the set of known activity signatures is stored in the first memory.

14. The system of claim 12, wherein the set of known activity signatures comprises signatures for running, walking, and being motionless.

15. The system of claim 12, wherein the set of known activity signatures comprises signatures for walking, running, sleeping, resting, changing elevation, turning, swimming, and riding in a vehicle.

16. The device of claim 1, further comprising an interface module configured to be worn about the user's body, and configured to audibly present the instruction to the user.

17. The system of claim 1, wherein the sensor module, the body area module, the activity module, and the interface module are all saved on a memory of the activity tracking device.

18. The system of claim 1, wherein the body area module is configured to prefer one context over another by a user.

19. The system of claim 1, wherein the device has no operating system, and function calls of the activity module are limited to function calls to drivers.
